(19) United States
(12) Patent Application Publication
(10) Pub. No.: US 2025/0152480 A1
(43) Pub. Date: May 15, 2025

(54) Emulsion Deodorant Compositions

(71) Applicant: Conopco, Inc., d/b/a UNILEVER, Englewood Cliffs, NJ (US)

(72) Inventors: Tammy Baskerville, Leeds (GB); Natasha Suzanne Rigby Day, Leeds (GB); Joy Rachel McWalter, Leeds (GB)

(21) Appl. No.: 18/943,458

(22) Filed: Nov. 11, 2024

(30) Foreign Application Priority Data
Nov. 13, 2023 (EP) 23209554.7

(51) Int. Cl. A61K 8/06; A61K 8/58; A61Q 15/00

(52) U.S. Cl. CPC A61K 8/062; A61K 8/58; A61Q 15/00; A61K 2800/48; A61K 2800/5422; A61K 2800/592; A61K 2800/805

(57) ABSTRACT

A rheologicaly stable oil-in-water emulsion deodorant composition comprising a nonionic emulsifier system; a deodorant active comprising zinc neodecanoate; and a rheology modifier consisting of a C16-C22 linear fatty alcohol and a C16-C22 fatty acid glyceride, wherein the composition has a viscosity of from 10,000 mPa·s to 300,000 mPa·s at 10/s and 20° C.

### FIELD OF THE INVENTION

The invention is concerned with water-in-oil emulsion deodorant compositions, particularly such compositions comprising zinc neodecanoate as a deodorant active and having excellent stability.

### BACKGROUND OF THE INVENTION

Emulsion deodorant compositions, including oi-in-water I emulsions are known in the art. The format of such compositions may typically be lotions, creams, or roll-ons.

Deodorant compositions comprising zinc-based actives are known in the art. Examples of publications disclosing such compositions are given below

Zinc ricinoleate is a particularly well known zinc-based deodorant. EP 3878431 A1 (Hyteck, 2021) discloses stable emulsion deodorant compositions comprising zinc ricinoleate.

WO 18/087147 A1 (Givaudan, 2018) discloses deodorant compositions comprising zinc neodecanoate and fragrance. It is mentioned therein that this active may be incorporated in a range of deodorant compositions; however, no issues concerning its incorporation into water-in-oil emulsions are mentioned.

WO 18/087148 A1 (Givaudan, 2018) discloses deodorant compositions comprising zinc neodecanoate and aluminium chlorohydrate.

### SUMMARY OF THE INVENTION

The invention is concerned with oil-in-water emulsion deodorant compositions comprising zinc neodecanoate as a deodorant active and having excellent stability.

It is an object of the invention to provide oil-in-water emulsion deodorant compositions having good efficacy and superior rheological stability.

In preferred embodiments, it is an object of the invention to provide oil-in-water emulsion deodorant compositions having good skin care properties.

In a first aspect of the invention, there is provided an oil-in-water emulsion deodorant composition comprising (i) a nonionic emulsifier system; (ii) zinc neodecanoate; and (iii) a rheology modifier consisting of a C16-C22 linear fatty alcohol and a C16-C22 fatty acid glyceride, wherein the composition has a viscosity of from 10,000 cPs to 300,000 cPs at 10/s and 20° C. In SI units, this equates to from 10,000 mPa·s to 3000,000 mP·s at 10/s and 20° C.

Herein, the abbreviation mP·s is short for the SI unit of viscosity millipascal·second.

In a second aspect of the invention, there is provided a method of manufacture of a deodorant composition according to the first aspect of the invention.

In a third aspect of the invention, there is provided a cosmetic method of obtaining a deodorancy benefit comprising the topical application to the surface of the human body of a deodorant composition according to the first aspect of the invention.

In a fourth aspect of the invention, there is provided the use of a deodorant composition according to the first aspect of the invention to deliver a deodorancy benefit to the surface of the human body.

### DETAILED DESCRIPTION OF THE INVENTION

Herein, features expressed as “preferred” with regard to a particular aspect of the invention should be understood to be preferred with regard to each aspect of the invention (likewise, features expressed as “more preferred”, “particularly preferred” or “most preferred”). “Preferred” features aid the delivery of one or more of the objects of the invention.

Herein, preferred features of the invention are particularly preferred when used in combination with other preferred features (likewise, features expressed as “more preferred”, “particularly preferred” or “most preferred”).

Herein, “ambient conditions” refer to 20° C. and 1 atmosphere pressure, unless otherwise indicated.

Herein, any material having a water solubility of less than 0.1 g/100 g at 20° C. under ambient conditions is considered to be water insoluble.

Herein, all percentages, ratios and amounts are by weight, unless otherwise indicated.

Herein, amounts and concentrations of ingredients are percentages by weight of the total composition, unless otherwise indicated.

Herein, references to amounts of components such as “oil” or “rheology modifier” relate to the total amount of such components present in the composition.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein, the “non-ionic emulsifier system” refers to all nonionic emulsifiers present in the composition and no other components.

Compositions of the invention comprise a non-ionic emulsifier system. Preferably, the nonionic emulsifier system comprises a low HLB ethoxylated alcohol (HLB less than 8) and a high HLB ethoxylated alcohol (HLB more than 12). More preferably, the low HLB ethoxylated alcohol has a HLB less than 5 and the high HLB ethoxylated alcohol has a HLB of more than 15.

When a nonionic emulsifier system as described in the paragraph immediately above is employed, it is preferred that the ratio of the low HLB ethoxylated alcohol to the high HLB ethoxylated alcohol is from 1:1 to 1:10 and more preferably from 2:3 to 1:8.

An especially preferred emulsifier nonionic system comprises Steareth-2 and Steareth-20. Steareth-2 has a HLB of 4.9 and Steareth-20 has a HLB of 15.3.

The non-ionic emulsifier system preferably comprises from 0.5 to 10%, more preferably from 1 to 8%, and most preferably from 1 to 6%, all relative to the total composition.

Zinc neodecanoate is used as a deodorant active used in the present invention comprises Zinc neodecanoate is the zinc dicarboxylate salt of neodecanoic acid having the formula: [C₉H₂₀-CO·O]₂Zn. Other deodorant actives may also be present, but zinc neodecanoate is essential.

The present inventors have found that there can be rheological stability issues with oil-in-water emulsions comprising zinc neodecanoate. A particular problem with such compositions is rheological instability, typically manifested by phase separation. The present invention addresses this problem.

The zinc neodecanoate is preferably present at from 0.1 to 20%, more preferably at from 0.5 to 10% and most preferably at from 1 to 7% by weight of the total composition.

By incorporating a rheological as described herein, rheologically stable oil-in-water emulsion compositions comprising zinc neodecanoate may be obtained.

The C16-C22 linear fatty alcohol is employed, it is preferably a C18 linear fatty alcohol and more preferable stearyl alcohol.

The C16-C22 linear fatty alcohol is preferably used at from 1 to 12% of the total composition and more preferably at from 2 to 7% by weight of the total composition.

The C16-C22 fatty acid glyceride is preferably rich in monoglyceride content, meaning that it comprises at least 25% C16-C22 fatty acid monoglyceride and more preferably at least 30% C16-C22 fatty acid monoglyceride.

The C16-C22 fatty acid glyceride preferably comprises at least 25% glyceryl monostearate and more preferably at least 30% of said monoglyceride.

The C16-C22 fatty acid glyceride is preferably used at from 1 to 12% of the total composition and more preferably at from 2 to 7% by weight of the total composition.

The ratio of the C16-C22 linear fatty alcohol to the C16-C22 fatty acid glyceride is preferably from 1:4 to 4:1, more preferably from 1:3 to 3:1 and most preferably from 1:2 to 2:1, by weight.

The total content of the rheological modifier consisting of a C16-C22 linear fatty alcohol and a C16-C22 fatty acid glyceride is preferably from 4 to 20%, more preferably from 5 to 15% and most preferably from 6 to 13%, by weight of the total composition.

Compositions of the invention are typically shear-thinning, i.e. they are non-Newtonian fluids that decrease in viscosity as the shear rate is increased. At a shear rate of /10 s, they have a viscosity of from 10,000 cPs to 300,000 cPs/s at 10/s, measured at 20° C. Preferably, they have a viscosity of from 25,000 cPs to 300,000 cPs/s at 10/s and more preferably a viscosity of from 50,000 cPs to 250,000 cPs/s at 10/s, all measured at 20° C.

Compositions of the invention are oil-in-water emulsions; hence, they comprise an oily phase dispersed in a continuous aqueous phase. The aqueous is typically the predominant phase, preferably comprising greater than 60%, more preferably greater than 75%, and most preferably greater than 80% by weight of the total composition.

The aqueous phase preferably comprises a humectant selected from glycerol and/or a PEG oligomer of weight average molecular weight of from 180 to 420. Such materials are water soluble. Glycerol is an especially preferred humectant. The humectant may enhance the skin care properties of the composition.

When employed, the humectant is present in the total composition at a preferred level of from 1 to 20%, more preferably from 2 to 12% and most preferably from 3 to 10%, these levels being particularly relevant when the humectant is glycerol.

In certain preferred embodiments, the aqueous phase comprises a water soluble vitamin, such as Vitamin B3 or niacinamide. Such vitamins are present at a level at a preferred level of from 0.01 to 6%, more preferably from 0.1 to 5% and most preferably from 1 to 5% by weight of the total composition. Such vitamins may enhance the skin care properties of the composition.

In certain preferred embodiments, the aqueous phase comprises an a-hydroxy acid, such as lactic acid. Such components are present at a level at a preferred level of from 0.01 to 6%, more preferably from 0.1 to 5% and most preferably from 1 to 5% by weight of the total composition. Such components may enhance the skin care properties of the composition.

The oily phase comprises water-insoluble hydrophobic material including at least one water-insoluble oil that is liquid at ambient temperature and pressure. The zinc neodecanoate deodorant active is also part of the oily phase.

The oily phase is typically the minor phase present in compositions of the invention, preferably comprising less than 40%, more preferably less than 30%, and most preferably less than 25% by weight of the total composition.

For the purpose of calculating the amounts of the aqueous and the oily phase, the nonionic emulsifier system and the rheology modifier are considered to be components of the oily phase.

The oily phase preferably comprises a fragrance oil, alternatively known as a perfume oil. The fragrance oil may comprise a single fragrance component or, more commonly, a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is preferably up to 3% advantageously is at least 0.5% and particularly preferably from 0.8% to 2%.

The water-insoluble oil that is liquid at ambient temperature and pressure may be any such oil or oils, including a fragrance oil as described above.

In certain embodiments, it is preferred to include an oil, other than a fragrance oil, that has a relatively low viscosity, by which is meant less 250 cS (mm^{2.}·s⁻¹). Such oils, termed “non-fragrance oils”, can improve the sensory properties of the composition on application and can lead to other benefits such as emolliency.

Preferred non-fragrance oils are alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Other preferred non-fragrance are triglyceride oils. The triglyceride oils commonly comprise the alkyl residues of aliphatic C₇ to C₁₈ acids, the total number of carbon atoms being selected in conjunction with the extent of olefinic unsaturation and/or branching to enable the triglyceride to be liquid at 20° C. Particularly preferably, in the triglyceride oil the alkyl residues are linear C18 groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid, petroselenic acid and stearidonic acid.

Suitable oils can include those derived from unsaturated C₁₈ acids, including coriander seed oil, impatiens balsimina seed oil, parinarium laurinarium kernel fat oil, sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil by virtue of its characteristics is sunflower (seed) oil.

Further suitable oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C₁₂₋₁₅ alkyl benzoate, for example Finsolv TN™ or Finsolv Sun™.

Other components that may be included in compositions according to the invention including those described in the following paragraphs.

Wash-off agents may be included, often in an amount of up to 10%, to assist in the removal of the formulation from skin or clothing. Such wash-off agents are typically non-ionic surfactants such as esters or ethers containing a C₈ to C₂₂ alkyl moiety and a hydrophilic moiety comprising a polyoxyalkylene group (POE or POP).

Other skin benefit agents, such as allantoin or lipids, may be included, typically in an amount of up to 5%.

A highly preferred optional component is a preservative, such as sodium benzoate/citric acid and/or an antioxidant such BHT (butyl hydroxy toluene), each typically in an amount of from 0.01 to 0.5%.

The method of manufacture of compositions of the invention involves the following steps.
A first vessel is filled with aqueous phase components, including water and any other water soluble components, such as a water soluble emollient, an alpha amino acid, vitamin(s) and/or preservative agent(s).
A second vessel is filled with the nonionic emulsifier system and the oil phase components including the zinc neodecanoate and a rheology modifier consisting of a C16-C22 linear fatty alcohol and/or a C16-C22 fatty acid glyceride.
Optionally but preferably, a third vessel is filled with further water and, optionally, fragrance encapsulates.
The first and second vessels are heated to a temperature of 75° C. or greater and the components are allowed to dissolve and homogenise, preferably with stirring.
The contents of the first vessel and second vessel are then mixed under shear, the contents of the second vessel being preferably added to the stirred contents of the first vessel, preferably into a vortex created in the contents of the first vessel by the stirring thereof.
Optionally but preferably, the contents of the third vessel are then added at no more than ambient temperature in order to quench (reduce) the temperature of the mixture described above.
Preferably, the mixture described above is cooled to less than 50° C. before fragrance is added and stirred in to emulsify.
Finally, the mixture is subjected to high shear in order to build its viscosity. A shear rate of at least 1500 rpm is preferred, more preferably at least 2500 rpm and most preferably at least 3500 rpm, in order to build its viscosity to from 10,000 mPa·s to 300,000 mPa·s at 10/s and 20° C.

### EXAMPLES

The following Examples are illustrative of the invention and non-limiting as to its scope. Examples according to the invention are indicated by numbers and Comparative Examples are indicated by letters.

Examples 1 and 2 illustrated in Table 1 were prepared by the following method. The term “batch” refers to the batch or sample of the mixture being prepared.

Into a main vessel, the following components were added: water, glycerol, niacinamide (Example 1 only) and sodium benzoate and lactic acid (Example 2 only). This mixture was heated to 85° C. and homogenised.

Into a first side-pot, the following components were added: the glyceryl stearate, stearyl alcohol, cetearyl alcohol (Example 2 only), steareth-2, steareth-20, PPG-15 stearyl ether (Example 1 only), antioxidant, and zinc neodecanoate. This mixture was also heated to 85° C. and homogenised.

Into a second side-pot, further water and phenoxyethanol and the fragrance encapsulates (Example 1 only) were placed.

The stir speed in the main vessel was increased to form a vortex and the contents of first side-pot were added into the vortex and the mixture was allowed to mix for sufficient time to give good emulsification throughout the batch. The contents of the second side-pot (at ambient temperature) were then added to start cooling the batch. The batch was cooled to 43° C.

The fragrance was then added and stirring continued for a further 5 minutes.

Finally, the batch was transferred to a high shear mixture and sheared at 3700 rpm for 5 minutes.

### Comparative Examples A to F were Prepared by Methods Known in the Art.

Regarding the raw materials:
The glyceryl stearate comprised 40% glyceryl monostearate.
“Antioxidant”=pentaerythrityl tetra-di-t-butyl hydrocinnamate.
The zinc neodecanoate also contained 20% isopropyl myristate.

TABLE 1

| | Examples | | | |
|---|---|---|---|---|
| | 1 | 2 | A | B |
| Glycerol | 3 | — | 4 | 4 |
| Glyceryl stearate | 3 | 7.5 | — | — |
| Stearyl alcohol | 5 | 3.5 | — | — |
| Cetearyl alcohol | — | 1 | — | — |
| Steareth-2 | 1 | 0.19 | 3 | 3.5 |
| Steareth-20 | 2 | 1.31 | 0.76 | 1.5 |
| PPG-15 stearyl ether | 3 | — | — | — |
| Sunflower seed oil | — | — | 4 | 0.5 |
| Antioxidant | 0.05 | — | 0.05 | 0.05 |
| Fragrance encapsulates | 0.75 | — | — | — |
| Zinc neodecanoate | 4 | 4 | 4 | 2.2 |
| Phenoxyethanol | 0.5 | — | 0.5 | — |
| Sodium benzoate | — | 0.5 | — | — |
| Disodium EDTA | — | — | 0.1 | 0.1 |
| Fragrance | 1.3 | 1.3 | 1 | 1 |
| Niacinamide | 3 | — | — | — |
| Lactic acid | — | 2.2 | — | — |
| Water | To 100 | To 100 | To 100 | To 100 |
| Viscosity (cPs) | 100k cPs | 250k cPs | Not | Not |
| | at 10/s | at 5/s | recorded | recorded |

All viscosities were measured at ambient temperature.

Examples 1 proved stable for 12 weeks at 45° C. and Example 2 was also stable. Comparative Examples A and B were not stable for 12 weeks at 45° C.—phase separation was observed well before this time.

The Comparative Examples in Table 2 also proved to be unstable.

TABLE 2

| | Examples | | | |
|---|---|---|---|---|
| | C | D | E | F |
| Glycerol | 4 | 4 | 4 | 4 |
| Steareth-2 | 3.5 | 3.5 | 3.5 | 3.2 |
| Steareth-20 | 1.5 | 0.9 | 1.5 | 0.9 |
| Sunflower seed oil | 1 | 1 | 2 | 2 |
| Xanthan gum | | 0.3 | 0.3 | 0.3 |
| Antioxidant | 0.05 | 0.05 | 0.05 | 0.05 |
| Zinc neodecanoate | 3 | 3 | 2 | 2 |
| Phenoxyethanol | — | — | — | — |
| Sodium benzoate | 0.5 | 0.5 | 0.5 | 0.5 |
| Disodium EDTA | 0.1 | 0.1 | 0.1 | 0.1 |
| Citric acid monohydrate | 0.5 | 0.5 | 0.5 | 0.5 |
| Fragrance | 1.1 | 1.1 | 1.1 | 1.1 |
| Water | To 100 | To 100 | To 100 | To 100 |
| Viscosity (cPs) at 10/s | 2650 | 3750 | 3820 | 4550 |

All viscosities were measured at ambient temperature. It will be noted that all of these are less than 10,000 cPs.

1. An oil-in-water emulsion deodorant composition comprising:
(i) a nonionic emulsifier system;
(ii) zinc neodecanoate; and
(iii) a rheology modifier consisting of a C16-C22 linear fatty alcohol and a C16-C22 fatty acid glyceride,
wherein the composition has a viscosity of from 10,000 mPa·s to 300,000 mPa·s at 10/s and 20° C.

2. The oil-in-water emulsion deodorant composition according to claim 1, wherein the C16-C22 fatty acid glyceride comprises at least 25% by weight of glyceryl monostearate.

3. The oil-in-water emulsion deodorant composition according to claim 1, wherein the C16-C22 linear fatty alcohol is stearyl alcohol.

4. The oil-in-water emulsion deodorant composition according to claim 1, wherein the C16-C22 fatty acid glyceride comprises at least 30% by weight of C16-C22 fatty acid monoglyceride.

5. The oil-in-water emulsion deodorant composition according to claim 1, wherein the nonionic emulsifier system comprises an ethoxylated alcohol of HLB less than 8 and an ethoxylated alcohol of HLB more than 12.

6. The oil-in-water emulsion deodorant composition according to claim 5, wherein the ethoxylated alcohol of HLB less than 8 has a HLB less than 5 and the ethoxylated alcohol of HLB more than 12 has a HLB of more than 15.

7. The oil-in-water emulsion deodorant composition according to claim 5, wherein the ratio of the ethoxylated alcohol with a HLB less than 8 to the ethoxylated alcohol having a HLB of more than 12 is from 1:1 to 1:10.

8. The oil-in-water emulsion deodorant composition according to claim 1, further comprising a humectant selected from glycerol and PEG oligomers having a molecular weight from 180 to 420.

9. The oil-in-water emulsion deodorant composition according to claim 1, wherein the C16-C22 linear fatty alcohol is present at from 2 to 10% by weight of the total composition.

10. The oil-in-water emulsion deodorant composition according to claim 1, wherein the C16-C22 fatty acid glyceride is present at from 1 to 6% by weight of the total composition.

11. The oil-in-water emulsion deodorant composition according to claim 1, further comprising a fragrance.

12. A method of making an emulsion deodorant composition, comprising
(a) a nonionic emulsifier system;
(b) zinc neodecanoate; and
(c) a rheology modifier consisting of a C16-C22 linear fatty alcohol and a C16-C22 fatty acid glyceride,
wherein the composition has a viscosity of from 10,000 mPa·s to 300,000 mPa·s at 10/s and 20° C.,
the method comprising:
(i) filling a first vessel with aqueous phase components, including water and any other water soluble components;
(ii) filling a second vessel with the nonionic emulsifier system and oil phase components including zinc neodecanoate and the rheology modifier consisting of a C16-C22 linear fatty alcohol and/or a C16-C22 fatty acid glyceride;
(iii) heating the first vessel and the second vessel to a temperature of 75° C. or greater, the components being allowed to dissolve and homogenise;
(iv) shear mixing a mixture of the contents of the first vessel and second vessel and
(v) subjecting the mixture to high shear of at least 1500 rpm in order to build its viscosity to from 10,000 mPa·s to 300,000 mPa·s at 10/s and 20° C.

13. The method according to claim 12, wherein the high shear rate referred to in step (v) is performed at 3500 rpm or greater.

14. A cosmetic method of obtaining a deodorancy benefit comprising topically applying the deodorant composition according to claim 1 to a surface of the human body.

15. (canceled)

16. The method according to claim 12, wherein step (iv) comprises adding the contents of the second vessel to the stirred contents of the first vessel to form the mixture.

17. The method according to claim 12, wherein step (ii) comprises the substep of filling a third vessel with water; and step (iv) comprises the substep of adding the contents of the third vessel to the mixture at no more than ambient temperature to reduce the temperature of the mixture.

18. The method according to claim 12, wherein step (iv) comprises the substep of cooling the mixture to less than 50° C. before adding fragrance.
